(19) United States
(12) Patent Application Publication
(10) Pub. No.: US 2025/0259698 A1
(43) Pub. Date: Aug. 14, 2025

(54) T-CELL RECEPTOR OPTIMIZATION USING QUANTUM VARIATIONAL AUTOENCODERS

(71) Applicant: NEC Laboratories America, Inc., Princeton, NJ (US)

(72) Inventor: Jonathan Warrell, Princeton, NJ (US)

(21) Appl. No.: 19/047,246

(22) Filed: Feb. 6, 2025

Related U.S. Application Data
(60) Provisional application No. 63/551,154, filed on Feb. 8, 2024.

(51) Int. Cl. G16B 15/30; G16B 40/30; G16H 20/17

(52) U.S. Cl. CPC G16B 15/30; G16B 40/30; G16H 20/17

(57) ABSTRACT

Systems and methods for t-cell receptor complex optimization using quantum variational autoencoders. Mixed-state t-cell receptor (TCR) embeddings and mixed-state major histocompatibility complex peptide (pMHC) embeddings can be generated by embedding input TCR sequences and input pMHC sequences, respectively, using a quantum variational autoencoder (QVAE). A combinatorial optimization of the mixed-state TCR embeddings while fixing the mixed-state pMHC embeddings can be performed using a machine learning-based predictor. TCR sequences from the mixed-state TCR embeddings and the mixed-state pMHC embeddings, after the combinatorial optimization, can be decoded using the QVAE to generate an optimized TCR sequence. The optimized TCR sequence can be synthesized as a synthetic compound for downstream tasks.

### RELATED APPLICATION INFORMATION

This application claims priority to U.S. Provisional App. No. 63/551,154, filed on Feb. 8, 2024, incorporated herein by reference in its entirety.

### BACKGROUND

### Technical Field

The present invention relates to quantum computing and more particularly to t-cell receptor (TCR) complex optimization using quantum variational autoencoders.

### Description of the Related Art

In the noisy intermediate-scale quantum era, quantum technologies are progressing rapidly. With this progress, classical machine learning methods are rapidly being generalized to operate in a quantum machine learning setting. However, the limited availability of quantum hardware and restrictions on the number of qubits in actual quantum devices underscores the need to minimize quantum resource requirements.

### SUMMARY

According to an aspect of the present invention, a computer-implemented method is provided, including, generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE), performing combinatorial optimization of the mixed-state TCR embeddings while fixing the mixed-state pMHC embeddings using a machine learning-based predictor, and decoding TCR sequences and peptide sequences from the mixed-state TCR embeddings and the mixed-state pMHC embeddings after the combinatorial optimization, using the QVAE to generate an optimized TCR sequence, and synthesizing a synthetic compound with the optimized TCR sequence for downstream tasks.

According to another aspect of the present invention, a system is provided, including, a memory device, one or more processor devices operatively coupled with the memory device to perform, generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE), performing combinatorial optimization of the mixed-state TCR embeddings while fixing the mixed-state pMHC embeddings using a machine learning-based predictor, and decoding TCR sequences from the mixed-state TCR embeddings and the mixed-state pMHC embeddings after the combinatorial optimization, using the QVAE to generate an optimized TCR sequence, and synthesizing a synthetic compound with the optimized TCR sequence for downstream tasks.

According to yet another aspect of the present invention, a non-transitory computer program product is provided including a computer-readable storage medium having a program code, wherein the program code when executed on a computer causes the computer to perform, generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE), performing combinatorial optimization of the mixed-state TCR embeddings while fixing the mixed-state pMHC embeddings using a machine learning-based predictor, and decoding TCR sequences from the mixed-state TCR embeddings and the mixed-state pMHC embeddings after the combinatorial optimization, using the QVAE to generate an optimized TCR sequence, and synthesizing a synthetic compound with the optimized TCR sequence for downstream tasks.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a flow diagram showing a high-level overview of a computer-implemented method for t-cell receptor (TCR) optimization using quantum variational autoencoders, is illustratively depicted in accordance with an embodiment of the present invention;

FIG. 2 is a block diagram showing an overall architecture of a quantum variational autoencoder (QVAE), in accordance with an embodiment of the present invention;

FIG. 3 is a flow diagram showing a method of training the QVAE, in accordance with an embodiment of the present invention;

FIG. 4 is a block diagram showing encoder and decoder circuits of the QVAE, in accordance with an embodiment of the present invention;

FIG. 5 is a block diagram showing an architecture of the QVAE with a quantum support vector classifier (QSVC), in accordance with an embodiment of the present invention;

FIG. 6 is a block diagram showing an operation of gate-based variational autoencoder, in accordance with an embodiment of the present invention;

FIG. 7 is a block diagram showing an operation of an annealing-based variational autoencoder, in accordance with an embodiment of the present invention;

FIG. 8 is a block diagram showing the relationships of the reconstruction loss, the regularization loss, and the classifier loss of the QVAE, in accordance with an embodiment of the present invention;

FIG. 9 is a block diagram showing a system implementing practical applications of t-cell receptor (TCR) complex optimization using quantum variational autoencoders, in accordance with an embodiment of the present invention; and

FIG. 10 is a block diagram showing a system for t-cell receptor complex optimization using quantum variational autoencoders, in accordance with an embodiment of the present invention.

### DETAILED DESCRIPTION OF PREFERRED EMBODIMENTS

In accordance with embodiments of the present invention, systems and methods are provided for t-cell receptor (TCR) complex optimization using quantum variational autoencoders.

In an embodiment, mixed-state t-cell receptor (TCR) embeddings and mixed-state major histocompatibility complex peptide (pMHC) embeddings can be generated by embedding input TCR sequences and input pMHC sequences, respectively, using a quantum variational autoencoder (QVAE). A combinatorial optimization of the mixed-state TCR embeddings while fixing the mixed-state pMHC embeddings can be performed using a machine learning-based predictor. TCR sequences from the mixed-state TCR embeddings and the mixed-state peptide embeddings, after the combinatorial optimization, can be decoded using the QVAE to generate an optimized TCR sequence. The optimized TCR sequence can be synthesized as a synthetic compound for downstream tasks.

Optimization of the binding between the T-cell receptor (TCR) and major histocompatibility complex peptide (pMHC) complexes is important in vaccine design, both for the purpose of developing vaccines for infectious diseases, and personalized treatments for cancer. The TCR is the molecule by which the immune system recognizes a particular protein (peptide). In the case of an infection, these are peptides from the infectious agent. For a cancer, they are peptides generated by the cancer (each are presented by the pMHC complex). Generating optimized TCR sequences for particular peptides is thus an important step in vaccine design, and requires a search over possible sequences, which is an exponentially large combinatorial search space. A similar problem arises in molecular design applications, such as small-molecule drug design and material design, where the search for optimal designs involves a search across a similar combinatorial space. Searching such spaces is difficult, since highly-efficient gradient descent techniques cannot be used directly (since the space is discrete), and there are potentially many local optima caused for instance by protein-folding energy landscapes.

Other approaches have performed optimization directly in the TCR sequence space or the latent representation of the TCR. In one approach, a reinforcement learning policy is learned to propose mutations to the original sequence which will improve the TCR binding to a target peptide according to a pretrained binding predictor. Alternatively, a continuous latent representation of TCR sequences is learned, and optimization of the TCR sequence is performed in the continuous latent space. Generation of candidates can be performed by directly manipulating the representation in the latent space according to semantics imposed during training, or applying a search-based optimization method to the latent space (e.g. gradient-descent or reinforcement learning) using a trained binding predictor which operates directly on the latent space.

The present invention can replace a classical continuous latent search space for molecular sequences with a quantum latent space, based on mixed quantum-states trained using a Quantum Variational Autoencoder. The Quantum Variational Autoencoder can be optimized using quantum analogues of the classical ELBO training bounds. The present invention can include molecular sequence data encoded initially as quantum pure states, and then compressed into a mixed-state encoding over a small number of qubits. The mixed-quantum encoding can be implemented either via a gate-based quantum circuit or a quantum annealing processor. The present invention can include a machine-learning based binding predictor, which can be a quantum classifier, or a classical model which takes a “classical shadow” of the latent state as inputs.

The advantages of the present invention over previous classical approaches include the ability of quantum states to compress exponential amounts of classical data (N qubits can encode 2^{N} classical bits), making them suitable for searching across exponential combinatorial spaces. Further, tunneling effects have been shown to offer advantages in searching such spaces through the ability to move between local optima that would be separated classically. Moreover, the use of quantum mixed-states makes the framework of the present invention well-suited to near-term quantum devices, by allowing intrinsic noise in quantum circuits to be incorporated into the model.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system, including a classical simulator, quantum gate-based, quantum annealing-based or hybrid computer. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

Referring now in detail to the figures in which like numerals represent the same or similar elements and initially to FIG. 1, a flow diagram showing a high-level overview of a computer-implemented method for TCR optimization using quantum variational autoencoders, is illustratively depicted in accordance with an embodiment of the present invention.

In an embodiment, mixed-state t-cell receptor (TCR) embeddings and mixed-state major histocompatibility complex peptide (pMHC) embeddings can be generated by embedding input TCR sequences and input pMHC sequences, respectively, using a quantum variational autoencoder (QVAE). A combinatorial optimization of the mixed-state TCR embeddings while fixing the mixed-state pMHC embeddings can be performed using a machine learning-based predictor. TCR sequences from the mixed-state TCR embeddings and the mixed-state peptide embeddings, after the combinatorial optimization, can be decoded using the QVAE to generate an optimized TCR. The optimized TCR sequence can be synthesized as a synthetic compound for downstream tasks.

In block 110, mixed-state t-cell receptor (TCR) embeddings and mixed-state major histocompatibility complex peptide (pMHC) embeddings can be generated by embedding input TCR sequences and input pMHC sequences, respectively, using a quantum variational autoencoder (QVAE).

The mixed state TCR embeddings can be generated by embedding input TCR sequences using the QVAE and the initial pure-state embeddings of the input TCR sequences. In another embodiment, the mixed state TCR embeddings can be generated by encoding the input TCR sequences using a quantum annealing processor such as a hierarchical quantum Boltzmann machine (h-QBM).

The initial pure-state embeddings of the input TCR sequences can be provided by a quantum data source. In another embodiment, the initial pure-state embeddings of the input TCR sequences can be generated by a data embedding circuit that can project the TCR sequences to a quantum state (e.g., amplitude encoding or angle embedding).

The mixed state pMHC embeddings can be generated by encoding the input pMHC sequences using the QVAE and the initial pure-state embeddings of the input pMHC sequences. In another embodiment, the mixed state pMHC embeddings can be generated by encoding the input pMHC sequences using a quantum annealing processor such as a hierarchical quantum Boltzmann machine (h-QBM).

The initial pure-state embeddings of the input pMHC sequences can be provided by a quantum data source. In another embodiment, the initial pure-state embeddings of the input pMHC sequences can be generated by a data embedding circuit that can project the pMHC sequences to a quantum state (e.g., amplitude encoding or angle embedding).

In block 120, a combinatorial optimization of the mixed-state TCR embeddings while fixing the mixed-state pMHC embeddings can be performed using a machine learning-based predictor.

The combinatorial optimization aims to search parameters where the mixed-state TCR embeddings can bind to the mixed-state pMHC embeddings. The combinatorial optimization can be performed with simultaneous perturbation stochastic approximation to perturb the parameters of an auxiliary quantum circuit that prepares a mixed state, which may have the same form as a QVAE encoder taking a predefined 0 state as input, to maximize the binding score (or a score representing a combination of desired properties as listed herein) of a quantum predictor such as a quantum support vector classifier (QSVC). In another embodiment the combinatorial optimization can be performed as described herein, but a classical shadow of the prepared TCR and pMHC mixed states may be used as inputs to a classical support vector classifier (SVC) or neural network to provide the binding score to be maximized.

In another embodiment, the combinatorial optimization can be performed using simultaneous perturbation stochastic approximation to perturb the parameters of a quantum annealer to produce a mixed state in an h-QBM, and a classical shadow of the prepared TCR and pMHC mixed states may be used as inputs to a classical support vector classifier (SVC) or neural network to provide the binding score to be maximized.

In block 130, TCR sequences from the mixed-state TCR embeddings and the mixed-state pMHC embeddings, after the combinatorial optimization, can be decoded using the QVAE to generate an optimized TCR sequence.

The optimized TCR sequence can include higher specificity, moderate affinity, structural complementarity (e.g., interface geometry, conserved docking orientation), thermodynamically balanced, balanced target functionality dynamics, autoimmunity avoidance, etc. These, and any other desired properties, can be reflected in the score used for combinatorial optimization of the TCR.

In block 140, the optimized TCR sequence can be synthesized as a synthetic compound for downstream tasks.

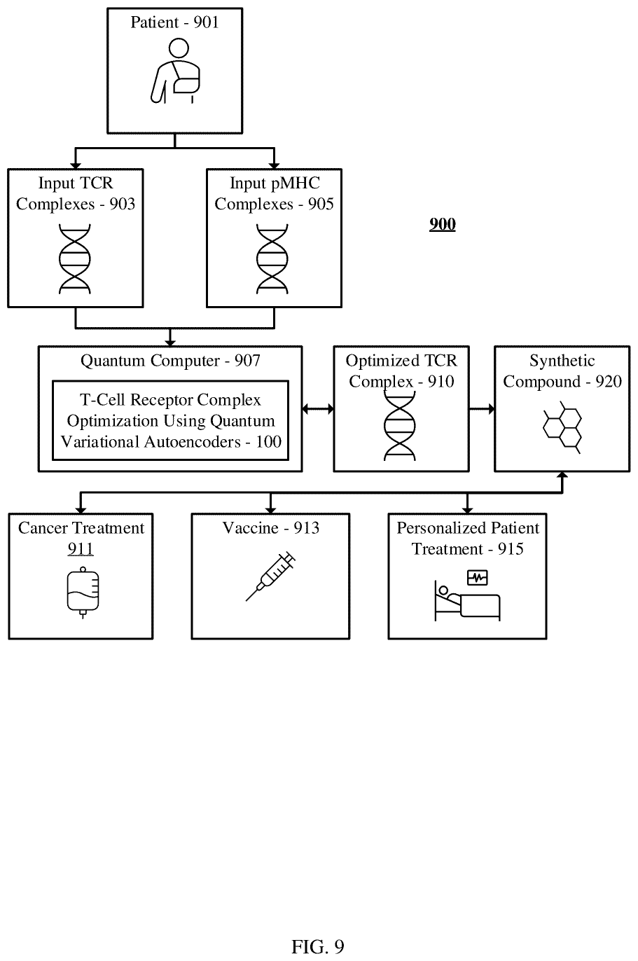

The optimized TCR complex can be synthesized as a synthetic compound having the desired properties that can be reflected in the score used for combinatorial optimization of the TCR. The synthetic compound can be synthesized to mimic TCR complexes. The optimized TCR sequence can be synthesized and used for several applications such as vaccine development, personalized patient treatments, and cancer treatment developments. The optimized TCR complex can be synthesized (e.g., single-cell ribonucleic acid (RNA) sequencing, retrovirus engineering, clustered regularly interspaced short palindromic repeats (CRISPR) targeted genome editing, etc.) to be usable for downstream applications such as cancer treatment, vaccine, and personalized patient treatment. Other synthesizing processes can be utilized. This is shown in more detail in FIG. 9.

Referring now to a description of the QVAE.

The QVAE can be implemented with a quantum computer. The quantum computer can include noisy intermediate-scale quantum (NISQ) machines that can have machine learning capabilities. The quantum computer may implement circuits to input amplitude-encoded state vectors, read out latent mixed states with sufficient accuracy and store resulting density matrices as classical shadows. Additionally, the quantum computer can include methods for divergence calculations between pairs of quantum states in the objective function and for quantum state tomography (e.g., matrix-state tomography, neural network-based tomography, etc.). The QVAE can also be simulated where latent states are represented by a small number of qubits. Other quantum devices can be used to implement the present invention such as IBM® Qiskit™ simulator, IBM® Quantum Heron™ gate-based system, Google® Willow™, D-Wave® Advantage™ 2 quantum annealer, etc.

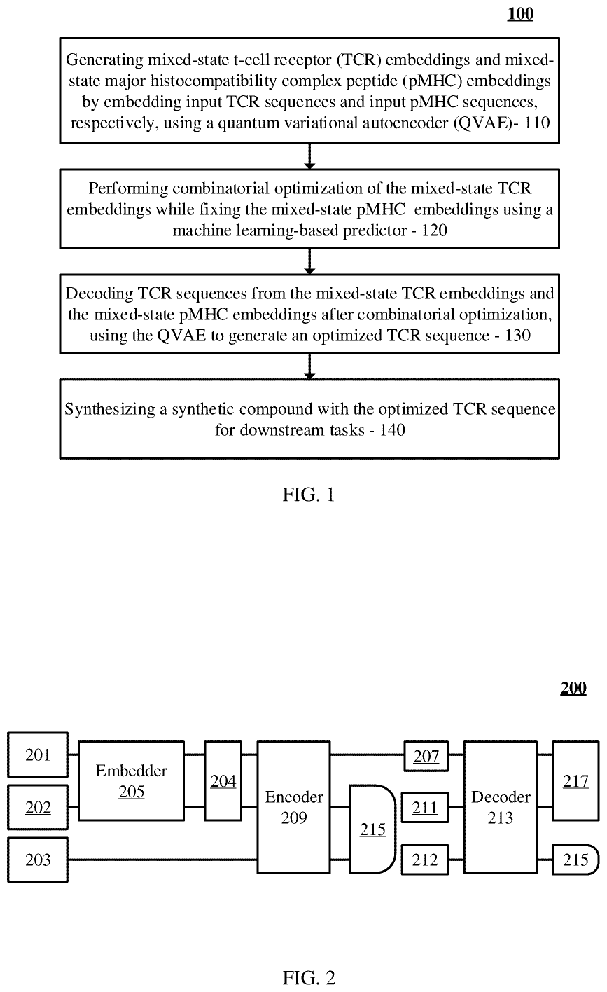

Referring now to FIG. 2, a block diagram showing an overall architecture of a quantum variational autoencoder (QVAE), in accordance with an embodiment of the present invention.

The QVAE 200 can include 2 input qubits (e.g., |0 [symbol] ) 201 and 202, a latent space 204 of 1 qubit and an auxiliary qubit 203 in both an encoder 209 and a decoder 213. Note that the specific number of qubits here is for illustration only, corresponding to the case where only 4 possible variants of the TCR and pMHC sequences are considered. In general, log₂ 20L qubits can be used, where L is the length of the TCR or pMHC sequence to be varied, where at each position one of 20 amino acids may appear.

The encoder 209 and decoder 213 can be defined by quantum circuits with trainable parameters θ_{E} and θ_{D} respectively. The corresponding unitary matrices for the encoder 209 and decoder 213 are denoted by U(θ_{E}) and V(θ_{D}) respectively. The embedder 205 performs the conversion from a classical source to a quantum representation unitary matrix Aᵢ for data-point i (e.g., using amplitude or angle embedding), where |ψᵢ [symbol] =Aᵢ|0 [symbol] , and ρᵢ=|ψᵢ [symbol] [symbol] ψᵢ|. After the embedding and encoding circuits have been applied to the initial |0 [symbol] state as in 211 and 212, both the N_{A} auxiliary qubits in 211 and the N_{T} “trash” qubits in 212 obtained by the encoder 209 can be discarded by a partial trace operation 215. The N_{T} “trash” qubits can be obtained as N_{T}=N_{X}−N_{Z}, N_{X} are the number of qubits in Hilbert space X, N_{Z} are the number of qubits in latent Hilbert space Z. The remaining qubit q1 is considered the latent state 207 ζᵢ. The output 317 of the QVAE is the reconstructed state σᵢ.

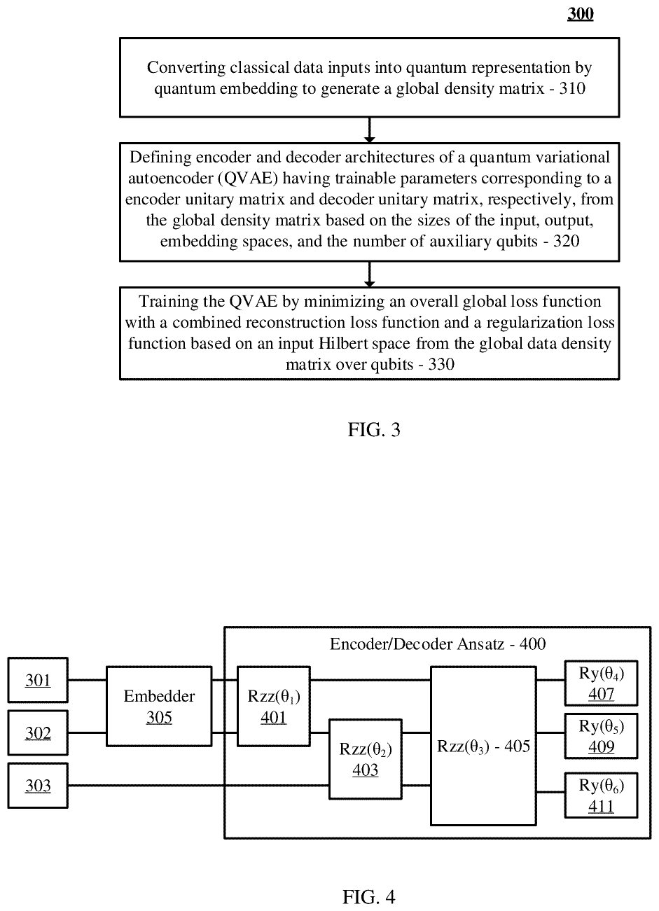

Referring now to FIG. 3, a flow diagram showing a method of training the QVAE, in accordance with an embodiment of the present invention.

In block 310, classical data inputs can be converted into quantum representation by quantum embedding (e.g., using amplitude or angle embedding) to generate a global density matrix.

In block 320, an encoder and decoder architecture for the QVAE having trainable parameters corresponding to an encoder unitary matrix and a decoder unitary matrix, respectively, taking the global density matrix as input, can be defined based on sizes of the input, output, embedding spaces, and the number of auxiliary qubits.

The number of auxiliary qubits can be chosen in the relation N_{B}=N_{X}+2N_{Z}, for maximal expressiveness. The number of auxiliary bits can be obtained by representing an arbitrary quantum channel T(⋅) between Hilbert spaces A and B with a unitary transformation U on A⊗B⊗C: T(ρ)=Tr_{AC}(U⁻¹(ρ⊗|ψ_{BC} [symbol] [symbol] ψ_{BC}|)U), where |ψ_{BC} [symbol] is an arbitrary pure state in B⊗C, Tr_{AC} denotes the trace over A⊗C, and C is an environment with dimension equal to the rank of the Choi matrix representation of T(⋅), using the Stinespring dilation. A final unitary permutation of the qubits can be appended, so that those of B are mapped to the initial qubits of A. An arbitrary channel between A and B can be represented by a Choi matrix of rank between 1 and dim(A)·dim(B)·dim(C) is at most 2^{N}^{_{X}}·2^{N}^{_{Z}} for input and output spaces X and Z respectively in an encoder (or Z and X in a decoder), where dim( ) is the dimension of a matrix. The Choi matrix can then be represented as log₂ N_{X}+N_{Z}=N_{X}+N_{Z} qubits in both encoder 209 and in decoder 213. Hence, U is over a space of dimension 2^{N}^{_{X}}·2^{N}^{_{Z}}·2^{N}^{_{X}}^{+N}^{_{Z}}=2^{2N}^{_{X}}^{+2N}^{_{Z}}, and the total number of auxiliary qubits in both encoder and decoder can then be N_{B}=N_{A}=(2N_{X}+2N_{Z})−N_{X}=N_{X}+2N_{Z}.

The QVAE 200 can define an encoder 209 and decoder 213 pair to compress an input dataset to Hilbert space, Z, over Nz qubits where the data is assumed in an input Hilbert space X, over Nx qubits. The input dataset can include a finite set of N initial pure-state embeddings |ψ₁ [symbol] . . . |ψ_{N} [symbol] which can be represented by a global density matrix ρ_{glob} having density matrices of individual datapoints ρᵢ obtained from an input dataset (e.g., input TCR sequences, input pMHC sequences, etc.):

$${ρ}_{glob} = \frac{1}{N} {\sum }_{i} {ρ}_{i} ; {ρ}_{i} = \vert {ψ}_{i} {ψ}_{i} \vert .$$

The QVAE 200 can learn quantum operations (completely positive trace-preserving (CPTP) linear maps) (E, D) corresponding to encoder 209 and decoder 213 respectively. The encoder 209 can have a signature: E: D(X)→D(Z) and the decoder 213 can have a signature: D: D(Z)→D(X), where D(X) denotes a set of density matrices over finite Hilbert space X, and D(Z) denotes a set of density matrices over finite Hilbert space Z, E∈S_{E} and D∈S_{D}, S_{E} and S_{D} are subsets of CPTP linear maps having a predefined maximum circuit complexity. The maximum circuit complexity can be set based on the characteristics of the device on which the system is implemented. For example, the IBM® Quantum Heron™ has a maximum complexity of approximately 5000 two-qubit gate operations.

Given the encoder 209 and decoder 213 pair, a latent state ζᵢ and reconstructed state σᵢ can be defined. Similarly, global latent state ζ_{glob} and global reconstructed state σ_{glob} can be defined. A predefined “prior” density matrix over the latent space ζ_{gen} can be assumed as

$${ζ}_{gen} = ( \frac{1}{{2}^{{N}_{z}}} ) {I}_{{2}^{{N}_{z}}} ,$$

where I is the identity operator. Similar to the classical case (e.g., non-quantum case), this prior can be transformed by the decoder 213 to produce a generative approximation of the data distribution σ_{gen}=D(ζ_{gen}).

The encoder 209 and decoder 213 can be defined by appending N_{A} auxiliary qubits to the input Hilbert space X and by appending N_{T} reference qubits with N_{B} auxiliary qubits to the latent Hilbert space Z, where N_{T}=N_{X}−N_{Z}, and N_{T} denotes the number of “trash” qubits.

The encoder 209 definition can be

$$E ( ρ ) = {Tr}_{{N}_{A} + {N}_{T}} ( {U}^{- 1} ( ρ ⊗ \vert {0}_{{N}_{A}} {0}_{{N}_{A}} \vert ) U ) ,$$

and the decoder 213 definition can be: D(ζ)=Tr_{N}_{_{B}}(V⁻¹(ζ⊗|0_{N}_{_{B}}_{+N}_{_{T}} [symbol] [symbol] 0_{N}_{_{B}}_{+N}_{_{T}}|)V), where U and V are unitary matrix representations of the encoder E and decoder circuits D, and Tr_{N}(⋅) denotes the trace over the final N qubits.

Referring now to FIG. 4, a block diagram showing encoder and decoder circuits of the QVAE, in accordance with an embodiment of the present invention.

Encoder/decoder Ansatz 400 can include quantum circuits 401, 403, 405, 407, 409 and 411. In quantum computing, an Ansatz is a wavefunction or state that can be used as a starting point for optimization or approximations.

Quantum circuit 401 can be a quantum gate that deals with a parametric 2-qubit Z⊗Z for parameter θ₁. Quantum circuit 401 can be a quantum gate that deals with a parametric 2-qubit Z⊗Z for parameter θ₁. Quantum circuit 403 can be a quantum gate that deals with a parametric 2-qubit Z⊗Z for parameter θ₂. Quantum circuit 405 can be a quantum gate that deals with a parametric 2-qubit Z⊗Z for parameter θ₃. Quantum circuit 407 can be a quantum gate that deals with a single qubit rotation about the Y axis for parameter θ₄. Quantum circuit 409 can be a quantum gate that deals with a single qubit rotation about the Y axis for parameter θ₅. Quantum circuit 411 can be a quantum gate that deals with a single qubit rotation about the Y axis for parameter θ₅.

Referring back to FIG. 3, in block 330, the QVAE can be trained by minimizing an overall global loss function with a combined reconstruction loss function and a regularization loss function based on an input Hilbert space from the global data density matrix over qubits.

To train the QVAE 200, a training loss can be derived. To derive the training loss, a model which minimizes a general loss (e.g., reconstruction loss) can be learned between the implicit generative model and the global data density matrix. The general loss can be non-negative and 0 if and only if a=b, but not necessarily symmetric. This can be expressed as:

$${min}_{D} {ℒ}_{1} ( {ρ}_{glob} , {σ}_{gen} ) .$$

To simultaneously learn a representation of data in the latent space, analogous to the classical variational autoencoders, a variational density parameterized by the encoder 209 (E) can be assumed to be expressive enough to fulfill the condition ζ_{glob}=ζ_{gen} can be expressed as:

$${min}_{D} {ℒ}_{1} ( {ρ}_{glob} , {σ}_{gen} ) = {{min}_{E , D}}_{s . t . {ζ}_{glob} = {ζ}_{gen}} {ℒ}_{1} ( {ρ}_{glob} , {σ}_{gen} ) .$$

This can be further reformulated as a constrained optimization problem by introducing a regularization loss [symbol] ₂ having the same conditions as

$${min}_{E , D} {ℒ}_{1} ( {ρ}_{glob} , {σ}_{gen} ) ; {ℒ}_{2} ( {ζ}_{glob} , {ζ}_{gen} ) \leq ϵ ; ϵ = {min}_{E} {ℒ}_{2} ( {ζ}_{glob} , {ζ}_{gen} ) .$$

To derive a training objective F for a global input density matrix ρ_{glob}, the Lagrange multiplier β≥0 is introduced, which is expressed as:

$${{min}_{E , D}}_{s . t . {ℒ}_{2} ( {ζ}_{glob} , {ζ}_{gen} ) \leq ϵ} {ℒ}_{1} ( {ρ}_{glob} , {σ}_{glob} ) \geq {max}_{β} {min}_{E , D} {F}_{glob} ( E , D , β ) ;$$

F_{glob}(E,D,β)= [symbol] ₁(ρ_{glob},σ_{glob})+β( [symbol] ₂(ζ_{glob},ζ_{gen})−ϵ)

β is treated as a hyperparameter when optimizing F_{glob} and the constant −βϵ is disregarded. When [symbol] ₁ and [symbol] ₂ are in quantum relative entropy, β=1 and ϵ=0, F_{glob}(E,D,B) forms an analogue of the classical evidence lower-bound (ELBO) in the classical variational autoencoder, which can be expressed in a generalized form as −S(ρ_{glob}|σ_{glob})≥−S(ρ_{glob}|σ_{glob})−S(ζ_{glob}|ζ_{gen}), where S(⋅|⋅) is the quantum relative entropy.

The generalized form can be obtained by assuming ρ_{glob}=Σᵢρᵢ|vᵢ [symbol] [symbol] vᵢ|,

$${ζ}_{gen} = ( \frac{1}{{2}^{{N}_{z}}} ) {\sum }_{j} \vert {w}_{j} 〉 〈 {w}_{j} \vert ,$$

and ζ_{glob}=E(ρ_{glob})=Σⱼqⱼ|wⱼ [symbol] [symbol] wⱼ|. The ζ_{gen} can be expressed in the same basis as ζ_{glob} because the former is the maximally mixed state which diagonalizes in any basis. −S(ρ_{glob}|σ_{glob}) can be expressed as:

−S(ρ_{glob}|σ_{glob})=Tr{ρ_{glob} log σ_{gen}}+S(ρ_{glob})=ΣᵢpᵢTr{|vᵢ [symbol] [symbol] vᵢ|log σ_{gen}}+S(ρ_{glob}).

Tr{|vᵢ [symbol] [symbol] vᵢ|log σ_{gen}} can be expressed as

$$Tr \{ \vert {v}_{i} 〉 〈 {v}_{i} \vert {log σ}_{gen} \} = {𝔼}_{j ∼ Categ ( 1 / {2}^{{N}_{z}} )} [ Tr \{ \vert {v}_{i} 〉 〈 {v}_{i} \vert D ( \vert {w}_{j} 〉 〈 {w}_{j} \vert ) \} ] = {𝔼}_{j ∼ Categ ( {q}_{1} ... {q}_{{2}^{{N}_{z}}} )} [ Tr \{ \vert {v}_{i} 〉 〈 {v}_{i} \vert D ( \vert {w}_{j} 〉 〈 {w}_{j} \vert ) \} \cdot \frac{{2}^{- {N}_{z}}}{{q}_{j}} ] = Tr \{ \vert {v}_{i} 〉 〈 {v}_{i} \vert {𝔼}_{j ∼ Categ ( {q}_{1} ... {q}_{{2}^{{N}_{z}}} )} [ D ( \vert {w}_{j} 〉 〈 {w}_{j} \vert ) \cdot \frac{{2}^{- {N}_{z}}}{{q}_{j}} ] \} ,$$

where Categ( ) denotes the categorical distribution, |vᵢ [symbol] and |wⱼ [symbol] are basis states in the input and latent space, defining bases in which ρ_{glob} and ζ_{glob} diagonalize respectively, qⱼ= [symbol] wⱼ|ζ_{glob}|wⱼ [symbol] , and [symbol] is the expectation over multiple repeated measurements.

By applying Jensen's trace inequality it can be expressed as:

$$Tr \{ \vert {v}_{i} 〉 〈 {v}_{i} \vert {log σ}_{gen} \} = Tr \{ \vert {v}_{i} 〉 〈 {v}_{i} \vert {𝔼}_{j ∼ Categ ( {q}_{1} ... {q}_{{2}^{{N}_{z}}} )} [ D ( \vert {w}_{j} 〉 〈 {w}_{j} \vert ) \cdot \frac{{2}^{- {N}_{z}}}{{q}_{j}} ] \} \geq Tr \{ \vert {v}_{i} 〉 〈 {v}_{i} \vert {𝔼}_{j ∼ Categ ( {q}_{1} ... {q}_{{2}^{{N}_{z}}} )} [ log D ( \vert {w}_{j} 〉 〈 {w}_{j} \vert ) \cdot \frac{{2}^{- {N}_{z}}}{{q}_{j}} ] \} = Tr \{ \vert {v}_{i} 〉 〈 {v}_{i} \vert {𝔼}_{j ∼ Q} [ log D ( \vert {w}_{j} 〉 〈 {w}_{j} \vert ) ] \} - {𝔼}_{j ∼ Q} [ log {q}_{j} ] + log {2}^{- {N}_{z}} = Tr \{ \vert {v}_{i} 〉 〈 {v}_{i} \vert {log σ}_{glob} \} + S ( {ζ}_{glob} ) - S ( {ζ}_{gen} ) .$$

Thus, through substitution and summing across i, −S(ρ_{glob}|σ_{glob})≥Σᵢρᵢ(Tr{|vᵢ [symbol] [symbol] vᵢ|log σ_{glob}}+S(ζ_{glob})−S(ζ_{gen}))+S(ρ_{glob})=−S(ρ_{glob}|σ_{glob})+S(ζ_{glob})−S(ζ_{gen}).

The training objective function F thus optimizes

$${min}_{D} {ℒ}_{1} ( {ρ}_{glob} , {σ}_{gen} )$$

when either, (a) both [symbol] ₁ and [symbol] ₂ are in quantum relative entropy with β=1, or (b) β=β*, where β* is the optimum value of β in

$${max}_{β} {min}_{E , D} {F}_{glob} ( E , D , β ) .$$

The reconstruction loss [symbol] ₁ can be quantum relative entropy which can be expressed as:

$${ℒ}_{1} ( ρ , σ ) = S ( ρ \vert σ ) = S ( ρ , σ ) - S ( ρ ) = - Tr ( ρlog ( σ ) ) - S ( ρ ) ;$$

$$where S ( ρ ) = - Tr ( ρlog ( ρ ) ) and S ( ρ , σ ) = - Tr ( ρlog ( σ ) ) .$$

The regularization loss [symbol] ₂ can be quantum relative entropy which can be expressed as:

$${ℒ}_{2} ( ζ , {ζ}_{gen} ) = S ( ζ , {ζ}_{gen} ) - S ( ζ ) = Tr ( ζlogζ ) - log \frac{1}{λ} = - S ( ζ ) + c ,$$

where

$$c = - log \frac{1}{λ} , ζ$$

is a mixed state latent representation and the analog of the classical generative “prior” on the latent space, ζ_{gen}. In another embodiment, the regularization loss can be fidelity loss, symmetric quantum relative entropy, etc.

The overall training objective can also be expressed as:

$${ℒ}_{glob} ( {θ}_{E} , {θ}_{D} , β ) = {ℒ}_{1} ( {ρ}_{glob} , {σ}_{glob} ) + β ( {ℒ}_{2} ( {ζ}_{glob} , {ζ}_{gen} ) ) .$$

Referring now to FIG. 5, a block diagram showing an architecture of the QVAE with a quantum support vector classifier (QSVC), in accordance with an embodiment of the present invention.

In system 500, the architecture of the QVAE can include a QSVC 510 which takes in an embedded density matrix generated by embedder 305 from input qubits 501, 502, and 503 and processed with the same Ansatz as the encoder/decoder Ansatz 400. The output of the encoder/decoder Ansatz 400 can then be processed by the QSVC 510.

The QSVC 510 can include a similarity kernel that can be obtained as the quantum fidelity between each pair. The QSVC can be trained using the loss, [symbol] ₃(ζ,l)=| [symbol] [y|ζ]−l|, where l is a target binary label (such as 1 or 0 for binding/non-binding), ζ is the latent representation of the TCR-pMHC pair, y is the predicted label as determined by measuring the output qubit of the QSVC, and [symbol] [⋅] is the expectation over multiple repeated measurements.

The overall system can be trained in multiple ways. In an embodiment, two separate QVAEs may be trained independently for the TCR and pMHC sequences, both using [symbol] _{glob-QVAE}. The latent embeddings produced by the separate encoders may then be used to train a QSVC using [symbol] ₃, where the inputs for the SVC are from the tensor product of the latent Hilbert spaces from the TCR and pMHC QVAE models. After optimizing the latent TCR representation for a desired pMHC, the output TCR representation may be generated by passing this latent representation through the TCR decoder.

In another embodiment, a single QVAE which jointly embeds TCR and pMHC pairs may be jointly trained with a QSVC. Here, the encoder Ansatz shown in 400 can be modified so that no interactions (e.g., no 2-qubit gates) are permitted between qubits taking input from the TCR sequence and those taking input from the pMHC sequence. However, such interactions may be present in the decoder Ansatz, as in 400. Qubits taking each kind of input can be retained in the latent space; hence those corresponding to pMHC inputs may be fixed when performing combinatorial optimization. The global loss for training using this second approach may be summarized as:

$${ℒ}_{QVAE + QSVC} ( {θ}_{E} , {θ}_{D} , β ) = {ℒ}_{1} ( {ρ}_{glob}^{TCR} , {σ}_{glob}^{TCR} ) + β ( {ℒ}_{2} ( {ζ}_{glob} , {ζ}_{gen} ) ) + γ {\sum }_{i} {ℒ}_{3} ( {ζ}_{i} , {l}_{i} ) ,$$

where γ is a weighting hyperparameter for the classifier loss, ζᵢ and lᵢ are the latent embedding and binary binding label respectively of datapoint i, and ρ_{glob}^{TCR} and σ_{glob}^{TCR} are the input and output global density matrices respectively of the TCR sequences (corresponding to a partial trace across the pMHC space of ρ_{glob} and σ_{glob} respectively).

Referring now to FIG. 6, a block diagram showing an operation of gate-based variational autoencoder, in accordance with an embodiment of the present invention.

A parameter 601 can be found which optimizes the TCR latent representation for the target pMHC by optimizing [symbol] ₃, representing a configuration of quantum gates (e.g., Rzz, Ry, etc.) using an Ansatz such as 400. The parameter 601 learned can then be processed by quantum gates to compute a latent representation 603. The latent representation 603 can then be processed by quantum gates (e.g., decoder) to compute an output state 605 for the optimized TCR.

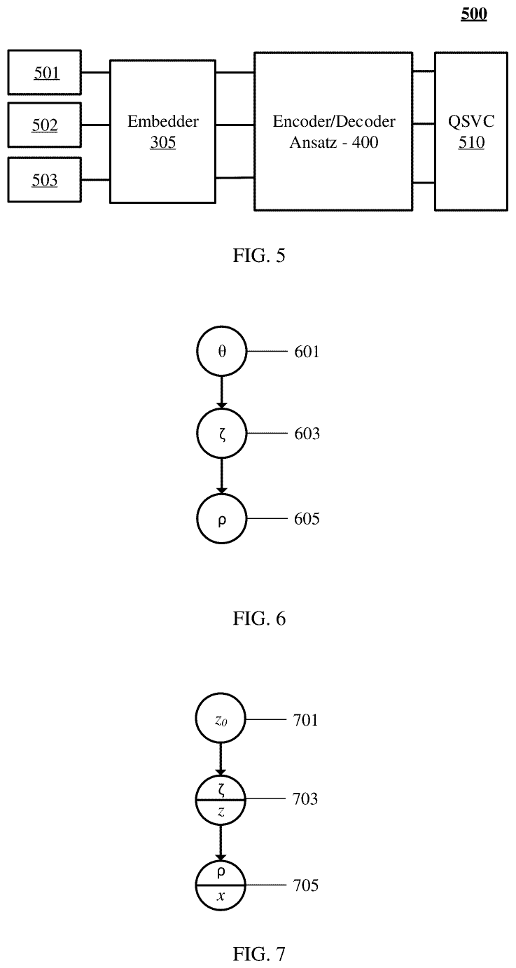

Referring now to FIG. 7, a block diagram showing an operation of an annealing-based variational autoencoder, in accordance with an embodiment of the present invention.

An initial state 701 can represent the parameters of a Quantum Boltzmann Machine (QBM) (e.g. the local field strengths, qubit couplings and transverse field strengths), and in 703, classical samples z from the mixed state ζ corresponding to the QBM may be generated using a quantum annealer. These in turn can fix the parameters in 705 of a QBM representing the output mixed state ρ, and classical samples x from ρ may be generated again using a quantum annealer. The initial state 701 can be optimized using [symbol] ₃, which here can be calculated over classical samples drawn from ζ (corresponding to TCR and pMHC pairs). The architecture in FIG. 7 may be referred to as a hierarchical Quantum Boltzmann Machine (hQBM), which realizes a hybrid quantum-classical system, and the same training methods as described herein may be applied, where the encoder architecture is a classical neural network, the decoder architecture is as shown in FIG. 7, a classical SVC is used as the classifier, and the classical KL-divergence is used in [symbol] ₁ and [symbol] ₂, with respect to a standard normal prior over the variable z₀.

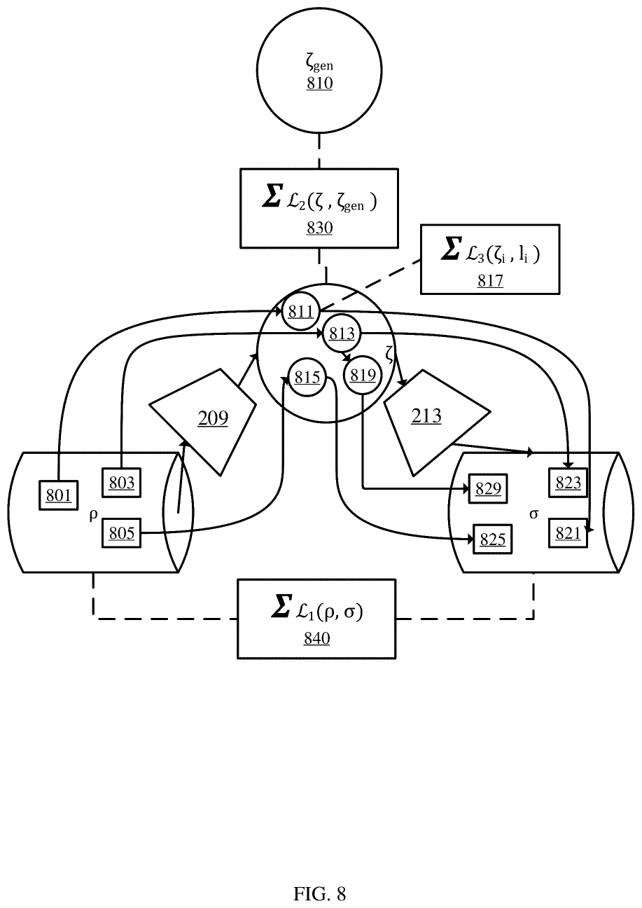

Referring now to FIG. 8, a block diagram showing the relationships of the reconstruction loss, the regularization loss, and the classifier loss of the QVAE, in accordance with an embodiment of the present invention.

Density matrices 801, 803 and 805 can be processed by encoder 209 with maximally mixed state 810 to generate mixed-state representations 811, 813, and 815, respectively, with regularization loss 830. The mixed-state representations 811, 813, and 815 can determine the QSVC loss 817 according to how well they predict the datapoint labels, lᵢ. Each mixed state representation represents the interaction of a TCR and pMHC pair. Hence, for a target pMHC complex, an optimal mixed state 819 can be generated which optimizes the classifier score, while fixing the parts of the mixed state corresponding to the desired pMHC complex. The optimal mixed state 819 is searched by performing combinatorial optimization, which may be initialized from a known example having the same pMHC sequence (such as 813). The mixed-state representations including 811, 813, 815, and 819 can be decoded using decoder 213 to generate reconstructed states 821, 823, 825, and 829, respectively, with reconstruction loss 840.

Referring now to FIG. 9, a block diagram showing a system implementing practical applications of t-cell receptor (TCR) complex optimization using quantum variational autoencoders, in accordance with an embodiment of the present invention.

In system 900, patient 901 can be diagnosed where targeted information about the disease of patient 901 (e.g., cancer, rare genetic diseases, etc.) can be identified having relevant input TCR complexes 903 and input pMHC complexes 905. A quantum computer 907 can implement TCR complex optimization using quantum variational autoencoders 100 to identify optimized TCR complex 910 having desired properties.

The optimized TCR complex 910 can be synthesized as a synthetic compound 920 through single-cell ribonucleic acid (RNA) sequencing, retrovirus engineering, clustered regularly interspaced short palindromic repeats (CRISPR) targeted genome editing, etc. to be usable for downstream applications such as cancer treatment 911, vaccine 913, and personalized patient treatment 915. Other synthesizing processes can be utilized.

To develop cancer treatment 911, the input TCR complexes 903 and input pMHC complex 905 can be identified and processed to target cancer cells. The optimized TCR complex 910 can be synthesized as a synthetic compound 920. The synthetic compound 920 can mimic antibodies that can include T-cells. T-cells can recognize antigens (e.g., MHC-peptide) on abnormal cells (e.g., cancer cells) and can be expressed as TCRs. The TCR can bind to the antigen and the T-cell can release toxic chemicals that can destroy the antigens. The cancer treatment 911 can be provided intravenously, orally, or with other appropriate administration route. Examples of cancer treatment 911 can include chimeric antigen receptor (CAR) T-cell therapy (e.g., Tisagenlecluecel), CAR natural killer (NK) cell therapy, etc.

To develop a vaccine 913, the input TCR complexes 903 and input pMHC complex 905 can be identified and processed to target infectious diseases such as influenza, tuberculosis, etc. The optimized TCR complex 910 can be synthesized as a synthetic compound 920. The synthetic compound 920 can mimic antibodies that can include T-cells. T-cells have TCRs that can recognize pathogens (e.g., having a pMHC) such as viruses, bacteria, fungi, and parasites). The TCR can bind to the antigen and the T-cell can release toxic chemicals that can destroy the pathogens. The vaccine 913 can be provided to the patient 901 intravenously, orally, or with other appropriate administration route. Examples of vaccine 913 can include protein-based vaccines such as conjugate vaccines (e.g., for pneumonia such as pneumococcal conjugate vaccine, meningitis, etc.), recombinant protein vaccines (e.g., for shingles, hepatitis B, etc.), polysaccharide vaccines (e.g., for pneumonia, meningitis, etc.), etc.

To develop personalized patient treatment 915, the input TCR complexes 903 and input pMHC complex 905 can be identified and processed to target the illness of patient 901. The optimized TCR complex 910 can be synthesized as a synthetic compound 920. The synthetic compound 920 can mimic antibodies that can include T-cells. T-cells can recognize antigens (e.g., pMHC) on abnormal cells that causes the illness of patient 901 (e.g., abnormal cells caused by an abnormal mutation in healthy cells) and can be expressed as TCRs. The TCR can bind to the antigen and the T-cell can release toxic chemicals that can destroy the antigens. The personalized patient treatment 915 can be provided to the patient 901 intravenously, orally, or with other appropriate administration route. Examples of personalized patient treatment 915 can include chimeric antigen receptor (CAR) T-cell therapy (e.g., Tisagenlecluecel), gene therapy drug for atopic dermatitis (e.g., abrocitinib), gene therapy drug for hemolytic anema (e.g., mitapivat) etc.

In another embodiment, the system 900 can also perform other downstream tasks such as classification of objects, data anomaly detection (e.g., performing combinatorial optimization on a data source to detect anomalous data based on learned “normal” data distributions and behavior), object identification, scene reconstruction, autonomous vehicle trajectory generation (e.g., finding the optimal route within a traffic scene), etc.

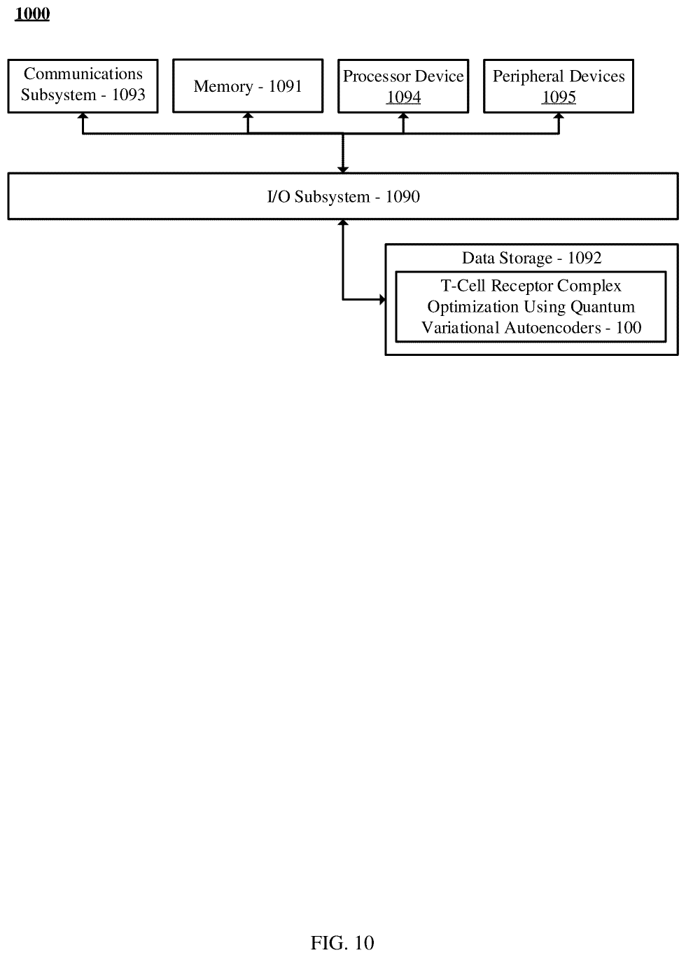

Referring now to FIG. 10, a block diagram showing a system for t-cell receptor complex optimization using quantum variational autoencoders, in accordance with an embodiment of the present invention.

The computing device 1000 illustratively includes the processor device 1094, an input/output (I/O) subsystem 1090, a memory 1091, a data storage device 1092, and a communication subsystem 1093, and/or other components and devices commonly found in a server or similar computing device. The computing device 1000 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 1091, or portions thereof, may be incorporated in the processor device 1094 in some embodiments. Further, components capable of implementing quantum gate-based computations, quantum annealing, and/or hybrid computations involving quantum and classical operations may be incorporated.

The processor device 1094 may be embodied as any type of processor capable of performing the functions described herein. The processor device 1094 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 1091 may be embodied as any type of volatile or non-volatile memory or data storage capable of performing the functions described herein. In operation, the memory 1091 may store various data and software employed during operation of the computing device 1000, such as operating systems, applications, programs, libraries, and drivers. The memory 1091 is communicatively coupled to the processor device 1094 via the I/O subsystem 1090, which may be embodied as circuitry and/or components to facilitate input/output operations with the processor device 1094, the memory 1091, and other components of the computing device 1000. For example, the I/O subsystem 1090 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 1090 may form a portion of a system-on-a-chip (SOC) and be incorporated, along with the processor device 1094, the memory 1091, and other components of the computing device 1000, on a single integrated circuit chip.

The data storage device 1092 may be embodied as any type of device or devices configured for short-term or long-term storage of data such as, for example, memory devices and circuits, memory cards, hard disk drives, solid state drives, or other data storage devices. The data storage device 1092 can store program code for t-cell receptor complex optimization using quantum variational autoencoders 100. Any or all of these program code blocks may be included in a given computing system.

The communication subsystem 1093 of the computing device 1000 may be embodied as any network interface controller or other communication circuit, device, or collection thereof, capable of enabling communications between the computing device 1000 and other remote devices over a network. The communication subsystem 1093 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 1000 may also include one or more peripheral devices 1092. The peripheral devices 1092 may include any number of additional input/output devices, interface devices, and/or other peripheral devices. For example, in some embodiments, the peripheral devices 1092 may include a display, touch screen, graphics circuitry, keyboard, mouse, speaker system, microphone, network interface, and/or other input/output devices, interface devices, GPS, camera, and/or other peripheral devices.

For quantum computing, the computing device 1000 perform operations on qubits. Qubits are analogous to classical bits but can have superposition of states which represent multiple possibilities at once. Qubits can behave like classical bits and have values of either zero or one but can also have a weighted combination of zero and one at the same time.

Of course, the computing device 1000 may also include other elements (not shown), as readily contemplated by one of skill in the art, as well as omit certain elements. For example, various other sensors, input devices, and/or output devices can be included in computing device 1000, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing system 1000 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE);
performing combinatorial optimization of the mixed-state TCR embeddings while fixing the mixed-state pMHC embeddings using a machine learning-based predictor;
decoding TCR sequences from the mixed-state TCR embeddings and the mixed-state pMHC embeddings after the combinatorial optimization, using the QVAE to generate an optimized TCR sequence; and
synthesizing a synthetic compound with the optimized TCR sequence for downstream tasks.

2. The computer-implemented method of claim 1, wherein the downstream tasks further comprises developing a treatment for cancer using the synthetic compound.

3. The computer-implemented method of claim 1, wherein the downstream tasks further comprises developing a vaccine against infectious diseases using the synthetic compound.

4. The computer-implemented method of claim 1, further comprising learning a quantum variational autoencoder (QVAE) with a quantum computer by minimizing an overall global loss function with a combined reconstruction loss function based on input and output Hilbert spaces over qubits, and a regularization loss function based on a latent Hilbert space over qubits.

5. The computer-implemented method of claim 4, wherein the reconstruction loss function is a quantum relative entropy loss based on a density matrix over a reconstructed state.

6. The computer-implemented method of claim 4, wherein the regularization loss function is a quantum relative entropy loss based on a mixed state latent representation and an analog of a classical generative prior on a latent space.

7. The computer-implemented method of claim 1, wherein a mixed latent quantum state is realized using a quantum annealer and the combinatorial optimization is performed using the quantum annealer.

8. A system, comprising:
a memory device;
one or more processor devices operatively coupled with the memory device to perform:
generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE);
performing combinatorial optimization of the mixed-state TCR embeddings while fixing the mixed-state pMHC embeddings using a machine learning-based predictor;
decoding TCR sequences from the mixed-state TCR embeddings and the mixed-state pMHC embeddings after combinatorial optimization, using the QVAE to generate an optimized TCR sequence; and
synthesizing a synthetic compound with the optimized TCR sequence for downstream tasks.

9. The system of claim 8, wherein the downstream tasks further comprises developing a treatment for cancer using the synthetic compound.

10. The system of claim 8, wherein the downstream tasks further comprises developing a vaccine against infectious diseases using the synthetic compound.

11. The system of claim 8, further comprising learning a quantum variational autoencoder (QVAE) with a quantum computer by minimizing an overall global loss function with a combined reconstruction loss function based on input and output Hilbert spaces over qubits, and a regularization loss function based on a latent Hilbert space over qubits.

12. The system of claim 11, wherein the reconstruction loss function is a quantum relative entropy loss based on a density matrix over a reconstructed state.

13. The system of claim 11, wherein the regularization loss function is a quantum relative entropy loss based on a mixed state latent representation and an analog of a classical generative prior on a latent space.

14. The system of claim 8, wherein a mixed latent quantum state is realized using a quantum annealer and the combinatorial optimization is performed using the quantum annealer.

15. A non-transitory computer program product comprising a computer-readable storage medium including a program code, wherein the program code when executed on a computer causes the computer to perform:
generating mixed-state t-cell receptor (TCR) embeddings and mixed-state major histocompatibility complex peptide (pMHC) embeddings by embedding input TCR sequences and input pMHC sequences, respectively, using a quantum variational autoencoder (QVAE);
performing combinatorial optimization of the mixed-state TCR embeddings while fixing the mixed-state pMHC embeddings using a machine learning-based predictor;
decoding TCR sequences from the mixed-state TCR embeddings and the mixed-state pMHC embeddings after combinatorial optimization, using the QVAE to generate an optimized TCR sequence; and
synthesizing a synthetic compound with the optimized TCR sequence for downstream tasks.

16. The non-transitory computer program product of claim 15, wherein the downstream tasks further comprises developing a treatment for cancer using the synthetic compound.

17. The non-transitory computer program product of claim 15, further comprising learning a quantum variational autoencoder (QVAE) with a quantum computer by minimizing an overall global loss function with a combined reconstruction loss function based on input and output Hilbert spaces over qubits, and a regularization loss function based on a latent Hilbert space over qubits.

18. The non-transitory computer program product of claim 17, wherein the reconstruction loss function is a quantum relative entropy loss based on a density matrix over a reconstructed state.

19. The non-transitory computer program product of claim 17, wherein the regularization loss function is a quantum relative entropy loss based on a mixed state latent representation and an analog of a classical generative prior on a latent space.

20. The non-transitory computer program product of claim 15, wherein a mixed latent quantum state is realized using a quantum annealer and the combinatorial optimization is performed using the quantum annealer.
